(19) United States
(12) Patent Application Publication
(10) Pub. No.: US 2011/0177175 A1
(43) Pub. Date: Jul. 21, 2011

(54) DIETARY FIBER COMPOSITIONS

(75) Inventors: Pratibha Sudhir Pilgaonkar, Mumbai (IN); Maharukh Tehmasp Rustomjee, Mumbai (IN); Anilkumar Surendrakumar Gandhi, Mumbai (IN); Rupali Kedar Suvarnapathaki, Mumbai (IN)

(73) Assignee: RUBICON RESEARCH PRIVATE LIMITED, mumbai (IN)

(21) Appl. No.: 13/120,339

(22) PCT Filed: Sep. 23, 2009
(86) PCT No.: PCT/IN09/00520

§ 371 Date: Mar. 22, 2011

(30) Foreign Application Priority Data
Sep. 23, 2008 (IN) 2031/MUM/2008

(51) Int. Cl. A61K 36/48; A61P 1/00; A23L 1/212

(52) U.S. Cl. 424/725; 426/615

(57) ABSTRACT

The present invention relates to dietary fiber compositions comprising fenugreek dietary fibers. The present invention also relates to use of these compositions as dietary supplements, functional foods or pharmaceutical preparations for promoting satiety and weight loss, lowering cholesterol, controlling blood glucose, maintaining colon health and modulating inflammation.

### FIELD OF THE INVENTION

The present invention relates to dietary fiber compositions comprising fenugreek dietary fibers. The present invention also relates to use of these compositions as dietary supplements, functional foods or pharmaceutical preparations for promoting satiety and weight loss, lowering cholesterol, controlling blood glucose, maintaining colon health and modulating inflammation. The present invention also provides processes of preparing compositions comprising fenugreek dietary fibers in the form of tablets, capsules, emulsions, suspensions, stick formulations, bars, powders or granules and the like for oral administration and methods of using them.

### BACKGROUND OF THE INVENTION

Dietary fiber is the part of a plant that provides and maintains its structure. Cellulose, hemicellulose, polysaccharides, pectins, gums, mucilages, and lignins are dietary fibers. Though these fibers are unrelated chemically, they all have one thing in common, which is that they are resistant to breakdown by the human digestive system (digestion). There are two major kinds of dietary fibers—insoluble (cellulose, hemicellulose, lignin) and soluble (gums, mucilages, pectins). Insoluble fibers are not digested by the body. They promote regularity and softening of stools. They also help to satisfy appetite by creating a full feeling. Soluble fibers are soluble in an aqueous enzyme system and delay the absorption of glucose into the blood stream preventing significant swings in blood sugar level and lower serum cholesterol. Modern day increase in the incidence and severity of diseases like hypertension, diabetes, colon cancer, irritable bowel syndrome and heart attacks have been linked to lack of fiber in the diet. Hence, nutritionists generally recommend 20 to 35 grams per day, or 10 to 13 grams per 1,000 kilocalories, intake of dietary fibers. Dietary fiber has a number of physiologically beneficial effects related to reduced risk of cancer and heart disease. Also, increased bulk from insoluble and soluble fiber contributes to a feeling of fullness, and because fiber is low in calories, this may help contribute to weight loss.

Sources rich in soluble fiber include oats, barley, rye, bananas, apples, berries, peas, fenugreek and soybeans and those rich in insoluble fiber include whole grains, whole wheat bran, nuts, seeds, green beans, carrots, celery, cauliflower and potatoes. Fenugreek or Trigonella foenum-graecum is one of the oldest known medicinal plants with dietary and medicinal uses. The most frequently used part of the fenugreek plant is the seed, which is ground and used as a spice in a variety of Indian, Middle Eastern and Mediterranean cuisines. It has also been traditionally used for its well-known carminative, galactogogue, antibacterial, anti-inflammatory and insulinotropic actions. The major constituents of fenugreek seeds have been identified as protein 20-25%, dietary fiber 45-50% having mucilaginous soluble fiber 15-30% and insoluble fiber 15-30%, fixed fatty acids and essential oils 6-8% and steroidal saponins 2-5%. Additionally, minor components like alkaloids (trigonolline, cholin, gentianine, carpaine, etc), free amino acids (4-hydroxyisoleucine) and individual spirostanols and furastanols like diosgenin, gitogenin, yamogenin, etc. have also been identified, isolated and characterized as principal components for its varying biological effects. Basch E et al., Alternative Medicine Review Volume 8, Number 1, 2003: 20-27 and Kumar K et al., Supplement to Agro FOOD industry hi-tech March/April 2008; 19:18-21 discuss the uses of fenugreek and fenugreek dietary fibers in further detail.

Various fenugreek seed extracts with varying concentrations of dietary fibers, soluble or insoluble, alkaloids, saponins or amino acids have been disclosed by researchers. U.S. Pat. No. 6,495,175 discloses a process for separating fixed oil and then oleoresin from fenugreek seeds to obtain a dietary fiber rich meal. These dietary fibers have a fat content of less than about 0.5%, fiber content in the range of about 48%-60% by weight or an average of about 52% by weight. It is also rich in proteins and well-provided with several vitamins and minerals. U.S. Pat. No. 5,997,877 provides a number of high-quality fractions of the fenugreek seed including a soluble dietary fiber fraction, de-flavored fenugreek seed, high-protein fenugreek meal, dioscin and other saponins, along with the fenugreek oleoresins which have conventional commercial use. Soluble fiber content in the soluble dietary fiber fraction was greater than 85%. PCT publications, WO 0128673A1 and WO 0174371A1 highlight fenugreek mucilages and galactomannan products and their use as thickeners, stabilizers or soluble fibers in food, cosmetic or pharmaceutical applications. U.S. Pat. No. 5,847,109 emphasizes emulsified isolated fenugreek galactomannan having at least 50 repeating units of mannose and galactose in a ratio of between 0.5-1.0 and 1.8-1.0, and consisting essentially of isolated hydrocolloid galactomannan fractions in combination with impurities selected from the group consisting of a protein content of less than 20 wt. %, a saponin content of less than 5 wt. % a lipid content of less than 1 wt. % and mixtures thereof, said galactomannan being present in an amount of up to 0.7 wt. % of the total emulsion. PCT publication WO 03094948 discloses novel compositions of bio-active compounds from fenugreek comprising between about 10% and about 70% of 4-hydroxyisoleucine and one or more compounds selected from the group of amino acids, alkaloids, glycosides, volatile oils, saponins, sapogenins, mannans, flavonoids, fatty acids, vitamins and provitamins, minerals, and carbohydrates. The disclosures, thus mainly provide Fenugreek extracts comprising varied amounts of different constituents. Some of these are rich in soluble fibers, some include in addition to soluble fibers constituents such as 4-hydroxyisoleucine or alkaloids or saponins or higher amounts of proteins. None of the disclosures, however emphasize upon isolation of fenugreek dietary fibers comprising mainly both insoluble and soluble dietary fibers.

EP1697050B1 (the '050 patent) teaches the process of isolation and purification of fiber rich fractions from fenugreek seeds to provide fiber rich fraction (FRF) and highly purified fiber rich fraction (HFRF). It discloses a unique process involving the first step of isolating the fiber rich fraction from the seeds by a mechanical process, followed by a purification step involving a hydroalcoholic process to obtain a highly purified fiber rich fraction from fenugreek seeds. Both FRF and HFRF have a combination of insoluble dietary fibers and soluble dietary fibers wherein the ratio of insoluble to soluble dietary fibers is greater than 0.8. The '050 patent enlists various properties and applications of these fibers mainly as excipients in the preparation of pharmaceutical compositions but it does not teach in any manner use of these fenugreek fibers or fiber rich fraction in effective amounts as a beneficial agent for promoting overall health and well being of a person.

Further, many researchers have tested the physiological effects of a variety of extracts from fenugreek, which are either soluble fiber enriched or contain 4-hydroxyisoleucine or saponins or alkaloids. In one study, the soluble dietary fiber fraction of Trigonella foenum-graecum (fenugreek) seed improves glucose homeostasis in animal models of type 1 and type 2 diabetes by delaying carbohydrate digestion and absorption, and enhancing insulin action (Hannan J. M. et al., Br. J. Nutr., March 2007, 97 (3):514-21). In another study, fenugreek seed extract, whose major component was 4-hydroxyisoleucine, was shown to reduce the body weight gain induced by a high-fat diet in obese mice. The extract decreased plasma triglyceride gain induced by oil administration (Handa T, et al.; Biosci. Biotechnol. Biochem, June 2005, 69 (6):1186-8). A study to determine the effects of ingesting an oral supplement containing 4-hydroxyisoleucine, isolated from fenugreek seeds, with a glucose beverage demonstrated that the rates of post-exercise glycogen resynthesis are enhanced above dextrose alone (Ruby B. C. et al.; Amino Acids, February 2005, 28 (1):71-6). Further, the soluble dietary fiber (SDF) fraction of Trigonella foenum graecum investigated for its chronic effect on serum fructosamine, insulin and lipid levels, and on platelet aggregation in Type 2 diabetic rats, demonstrated a beneficial effect on dyslipidemia and a tendency to inhibit platelet aggregation (Hannan J. M. et al., J, Ethnopharmacol., September 2003, 88 (1):73-7). In another study, defatted fractions of fenugreek seed were added to the diet of normal or diabetic hypercholesterolaemic dogs for 8 days. The defatted fraction rich in fibres (53.9%) and containing 4.8% of steroid saponins significantly lowered basal blood glucose, plasma glucagon and plasma cholesterol levels in normal dogs (Valette G. et al., Atherosclerosis, January 1984, 50 (1):105-11). In another study, the hypocholesterolaemic properties of an ethanol extract from defatted fenugreek (Trigonella foenum-graecum) seeds were investigated. Hypocholesterolaemic components, namely saponins were shown to interact with bile salts in the digestive tract (Stark A. et al., Br. J. Nutr., January 1993, 69 (1):277-87).

Thus, the research work with respect to fenugreek seeds discloses either processes of producing fenugreek seed extracts of diverse contents or the use of such extracts in various compositions, either alone or in combination with other bioactives for prevention or treatment of various diseases or disorders. Though a number of references highlight the use of fenugreek seed extracts, especially referring to soluble fiber enriched or saponin, alkaloid or 4-hydroxyisoleucine enriched extracts, either alone or in combination with other ingredients, none of the disclosures teach compositions or extracts providing mainly insoluble and soluble fibers from fenugreek for attaining desired physiological effects and promoting overall well-being of an individual. There thus exists a need to develop compositions that primarily comprise dietary fibers with an optimal amount of insoluble and soluble fibers which can synergistically aid in promoting the overall health and well-being of an individual by addressing issues of obesity, diabetes, cholesterol levels, colon health as well as inflammation. Further, it was surprisingly found that rather than utilizing insoluble and soluble dietary fibers from different fiber sources for synergistic benefits, dietary fibers obtained from fenugreek serve as a single source that provide an optimal amount and an optimal ratio of insoluble to soluble fibers for promoting overall health and well-being of an individual. Inventors have also described various compositions such as tablets, capsules, emulsions, suspensions, stick formulations, bars, powders or granules comprising such fenugreek dietary fibers.

### SUMMARY OF THE INVENTION

The present invention provides a dietary fiber composition comprising an effective amount of fenugreek dietary fiber. The present invention further relates to the use of these compositions as dietary supplements, functional foods or pharmaceutical preparations for promoting satiety and weight loss, lowering cholesterol, controlling blood glucose, maintaining colon health and modulating inflammation.

### OBJECTS OF THE INVENTION

It is an object of the present invention to provide dietary fiber compositions comprising effective amounts of Fenugreek dietary fibers comprising insoluble and soluble dietary fibers.

It is a further object of the present invention to provide compositions comprising fenugreek dietary fibers in the form of dietary supplements, functional foods or pharmaceutical preparations.

It is another object of the present invention to provide compositions comprising fenugreek dietary fibers for promoting satiety and weight loss, lowering cholesterol, controlling blood glucose, maintaining colon health and modulating inflammation.

It is another object of the present invention to provide compositions comprising fenugreek dietary fibers in the form of tablets, capsules, emulsions, suspensions, stick formulations, bars, powders or granules and the like for oral administration.

It is another object of the present invention to provide process of preparing compositions for oral administration comprising fenugreek dietary fibers.

It is still another object of the present invention to provide methods of using the compositions of the present invention comprising Fenugreek dietary fibers.

### DETAILED DESCRIPTION OF THE INVENTION

Regular dietary fiber intake is associated with many health benefits. Such intake needs to provide effective amounts of soluble and insoluble fibers, which can aid efficient absorption of vitamins and nutrients and also prevent several common health ailments that can become serious over time.

The present invention is directed to compositions comprising an effective amount of fenugreek dietary fibers. The term “effective amount” as used herein refers to an amount of fenugreek dietary fibers that is nontoxic but sufficient to provide the desired preventive, therapeutic and/or beneficial effects for promoting overall health and well-being of an individual, such as promoting satiety and weight loss, lowering cholesterol, controlling blood glucose, maintaining colon health and modulating inflammation. The term “fenugreek dietary fibers” as used herein refers to dietary fibers from fenugreek comprising soluble and insoluble fibers, which are further characterized by being substantially free of 4-hydroxyisoleucine, saponins and alkaloids and having a protein content of not more than about 10% by weight of the fiber.

The term “substantially free” as used herein means the fenugreek dietary fibers employed in the compositions of the present invention contain not more than 1% by weight, preferably not more than 0.4% of 4-hydroxyisoleucine, not more than 5%, preferably not more than 1%, more preferably not more than 0.5% of alkaloids such as trigonelline and not more than 5%, preferably not more than 1%, more preferably not more than 0.5% of saponins such as diosgenin.

The fenugreek dietary fibers incorporated in the compositions of the present invention comprise soluble and insoluble dietary fibers. In one embodiment of the present invention, the fenugreek fibers discussed in the '050 patent prepared from the husk of fenugreek seed rather than the endosperm and, therefore, being substantially free of 4-hydroxyisoleucine, saponins and alkaloids, and having a protein content of not more than about 10% by weight of the fiber, may be employed for the compositions of the present invention. The fenugreek fiber employed in the compositions of the present invention may in another embodiment have a viscosity greater than 10,000 cps at 2% w/v at 25° C. and protein content of less than 10% by weight of the fiber as described in the '050 patent.

The soluble dietary fiber is present in an amount from about 5% to about 95% by weight of the fenugreek fiber and the insoluble dietary fiber is present in an amount from about 5% to about 95% by weight of the fenugreek fiber. In one embodiment of the present invention the ratio of insoluble dietary fiber to soluble dietary fiber in the fenugreek fibers employed is about 0.2 to about 5. In another embodiment of the present invention, this ratio is about 0.5 to about 4. In yet another embodiment of the present invention, this ratio is about 0.8 to about 3. In a further embodiment, the ratio of insoluble dietary fiber to soluble dietary fiber in the fenugreek fibers employed is about 1 to about 3.

The dietary fiber compositions of the present invention comprise fenugreek fibers in an amount from about 5% to about 95% by weight of the composition. The compositions of the present invention may comprise from about 100 mg to about 20 g of fenugreek dietary fibers. In one embodiment the compositions may comprise from about 250 mg to about 15 g of fenugreek dietary fibers comprising both soluble and insoluble fibers for oral administration once, twice or thrice per day. Further, fenugreek fibers can be incorporated in the compositions of the present invention in any suitable form such as, but not limited to, powder or granules.

Without binding to any theory, the efficacy of the compositions of the present invention in maintaining health and overall well-being of a subject is based, amongst others, on the swelling, water holding and bile salt adsorption ability of the fenugreek fibers comprising both soluble and insoluble fibers incorporated therein. When hydrated, fenugreek dietary fibers employed in the compositions of the present invention form voluminous gels that bind to and retain portions of food ingested, resulting in a reduction of glucose absorption. Extensive gel formation in the intestine delays gastric emptying as well as slowly leaches glucose trapped in the gel, thereby preventing sudden rise in blood glucose levels, fatigue and overeating. It thus improves glucose homeostasis by delaying carbohydrate digestion and absorption and enhancing insulin action, resulting in better glucose tolerance. The fenugreek fibers employed in the compositions of the present invention with optimal amount of soluble and insoluble fibers thus enhance the food bulk, generate a feeling of satiety and promote weight loss. Further, the soluble dietary fibers from fenugreek dietary fibers are chemically galactomannans with a galactose to mannose ratio of 1:1. The galactose and mannose residues of the fenugreek dietary fibers are uniformly linked, thus providing maximum hydration and solubility.

Further, the swelling and water holding ability of the fenugreek fibers employed in the compositions of the present invention results in an increase in bulk, reduction in the transit time of the stool in the colon and helps prevent chronic constipation. Further, reduction in the transit time of stool prevents exposure to harmful toxins, such as faecal carcinogens and harmful colonic bacteria. The fenugreek dietary fibers serve as gentle and safe laxative which help reduce the risk of colorectal cancer and other bowel disorders like irritable bowel syndrome, diverticular disease and hemmoroids often associated with a low-fiber diet. Further, soluble fibers of fenugreek dietary fibers also undergo fermentation in the gut producing some short chain fatty acids which are important from the point of view of protection against colon cancer. Additionally, the viscous gels generated by the fenugreek dietary fibers incorporated in the compositions of the present invention help protect against inflammation. The soluble and insoluble fibers are also protective against C-reactive protein levels aiding in modulation of inflammation. The compositions of the present invention thereby aid in maintaining colon health and modulating inflammation.

The compositions of the present invention comprising fenugreek fibers can reduce cholesterol levels because the fenugreek fibers employed therein have a high ability to adsorb with bile salts. This results in increased conversion of hepatic cholesterol to bile salts due to loss, in the faeces, of complexes of these substances with fenugreek fiber. Thus, inhibition of reabsorption of bile salts in the distal intestine results in increased decomposition of cholesterol in the bile acid synthesis. Additionally, the short chain fatty acids produced by fermentation of soluble fibers in the colon act to reduce cholesterol synthesis. The mannose/galactose ratio of the fenugreek galactomannans provides exceptional surface properties at very low concentrations. These excellent interfacial properties are instrumental in managing the glucose response in diabetic patients, in reducing cholesterol levels and in the ability to efficiently entrap fat, thereby promoting weight loss.

The compositions of the present invention comprising fenugreek dietary fibers having optimal ratio of insoluble to soluble fibers act synergistically to provide the subject in need thereof health benefits such as promoting satiety and weight loss, lowering cholesterol, controlling blood glucose, maintaining colon health and modulating inflammation. Further, compositions of the present invention promote colon health, and reduce the risk of diabetes, intestinal problems, and heart disease. Fenugreek dietary fiber compositions of the present invention containing a combination of insoluble and soluble dietary fibers provide optimal effects with respect to control of blood glucose levels, lowering cholesterol, promoting weight loss, reducing obesity, maintaining colon health, modulating inflammation.

In an embodiment of the present invention, the dietary fiber compositions comprising effective amount of fenugreek dietary fiber can be in the form of dietary supplements, functional foods or pharmaceutical preparations that can promote health and overall well being of a subject by promoting satiety and weight loss, lowering cholesterol, controlling blood glucose, maintaining colon health and modulating inflammation.

The term “pharmaceutical preparation” refers to a preparation comprising effective amount of fenugreek dietary fibers formulated for therapeutic use. The term “dietary supplement” refers to a preparation or a formulation comprising effective amount of fenugreek dietary fibers used to supplement the diet of an animal or human to provide therapeutic, preventive and/or beneficial effects. The compositions of the present invention when in the form of pharmaceutical preparations or dietary supplement comprise in addition to the fenugreek dietary fibers, at least one pharmaceutically acceptable excipient.

At least one pharmaceutically acceptable excipient present in the pharmaceutical preparations or dietary supplements of the present invention includes, but is not limited to, fillers, binders, glidants, lubricants, stabilizers, solubilizers, disintegrants, polymers, sweeteners or flavourants. Fillers employed in the compositions include, but are not limited to, lactose monohydrate, microcrystalline cellulose or dicalcium phosphate. Binders employed in the compositions of the present invention include, but are not limited to, starch, pregelatinized starch, polyvinyl pyrrolidone (PVP), copovidone, cellulose derivatives, such as hydroxypropylmethyl cellulose (HPMC), hydroxypropyl cellulose (HPC) and carboxymethyl cellulose (CMC) and their salts or like. Glidants employed include, but are not limited to, colloidal silicon dioxide, talc, magnesium trisilicate, powdered cellulose, talc, tribasic calcium phosphate and the like. Lubricants employed in the compositions include, but are not limited to, colloidal silica, magnesium stearate, hydrogenated vegetable oils and triglycerides of stearic acid, palmitic acid or the like. The compositions of the present invention include at least one disintegrant such as, but not limited to, natural, modified or pregelatinized starch, crospovidone, croscarmellose sodium, sodium starch glycolate, low-substituted hydroxypropyl cellulose as well as effervescent disintegrating systems. The solubilizer includes, but is not limited to, hydrophilic surfactants or lipophilic surfactants or mixtures thereof. The surfactants may be anionic, nonionic, cationic, and zwitterionic surfactants. Examples of solubilizers include, but are not limited to cetostearyl alcohol, cholesterol, diethanolamine, ethyl oleate, ethylene glycol palmitostearate, glycerin, glyceryl monostearate, isopropyl myristate, lecithin, medium-chain glyceride, monoethanolamine, oleic acid, propylene glycol, polyoxyethylene alkyl ether, polyoxyethylene castor oil glycoside, polyethylene sorbitan fatty acid ester, polyoxyethylene stearate, propylene glycol alginate, sorbitan fatty acid ester, stearic acid, sunflower oil, triethanolmine, cyclodextrins and mixtures thereof. Examples of surfactants include, but are not limited to, sodium docusate, glyceryl monooleate, polyethylene alkyl ether, polyoxyethylene sorbitan fatty acid ester, sodium lauryl sulfate, sorbic acid, sorbitan fatty acid ester, and mixtures thereof. The tablet compositions of the present invention may also include stabilizers such as, but not limited to, benzoic acid, sodium benzoate, citric acid, and the like. Polymers employed in the compositions of the present invention include, but are not limited to, cellulose derivatives; polyhydric alcohols; saccharides, gums and derivatives thereof; vinyl derivatives, polymers, copolymers or mixtures thereof; maleic acid copolymers; polyalkylene oxides or copolymers thereof; acrylic acid polymers and acrylic acid derivatives; or any combinations thereof. Sweeteners employed in the compositions include, but not limited to, aspartame, stevia extract, glycyrrhiza, saccharine, saccharine sodium, acesulfame, sucralose and dipotassium glycyrrhizinate. Flavourants employed in the compositions include, but not limited to, mint flavour, orange flavour, lemon flavors, strawberry aroma, vanilla flavour, raspberry aroma, cherry flavor, tutty frutty flavor, magnasweet 135, key lime flavor, grape flavor, trusil art 511815, and fruit extracts.

In one illustrative embodiment according to the present invention, the dosage form may be optionally coated. The coating can be functional or non-functional or can be aqueous or non-aqueous. Further, the compositions of the present invention may be in the form immediate release, controlled/sustained/extended release or rapidly disintegrating such as orally disintegrating or chewable dosage form.

In one embodiment the pharmaceutical preparations of the present invention comprising fenugreek dietary fibers, may optionally comprise a pharmaceutically active agent that aids promoting satiety and weight loss, lowering cholesterol, controlling blood glucose, maintaining colon health and modulating inflammation.

Pharmaceutical preparations can be provided in the form of, but not limited to, tablets, capsules, emulsions, suspensions, powders, pellets or granules for oral administration or in sterile form for intravenous, intramuscular or subcutaneous administration. The pharmaceutical compositions may be administered to humans in a safe and effective amount to elicit desired results indicated for compositions of the present invention. In one embodiment of the present invention, the dosage form is a solid dosage form, for example, a tablet, which may vary in shape including, but not limited to, oval, triangle, almond, peanut, parallelogram, pentagonal. It is contemplated within the scope of the invention that the dosage form can be encapsulated. Tablets in accordance with the invention may be manufactured using conventional techniques of tableting methods known in the art such as direct compression, wet granulation, dry granulation or extrusion/melt granulation and can be monolithic or multilayered.

In another embodiment of the present invention the dietary fiber compositions of the present invention in the form of dietary supplements are meant for oral ingestion. Dietary supplements may also be administered by other routes including, but not limited to, nasally, sublingually, transmucosally, intragastrically or in a sterile manner intravenously, intramuscularly or subcutaneously. The route of administration can vary depending upon the therapeutic goals. The compositions of the present invention in the form of dietary supplements may be, for example, a liquid, solution, suspension, syrup, emulsion, pill, tablet, gel cap, capsule, powder, granules or pellets.

Further, in one embodiment the present invention also provides a process for preparing a composition in the form of a pharmaceutical preparation or a dietary supplement comprising blending fenugreek dietary fibers and at least one pharmaceutically acceptable excipient and compressing the blend to form tablets.

The term “functional foods” refers to are food substances, fresh or processed, comprising fenugreek dietary fibers that provide health-promoting and/or disease-preventing benefits beyond the basic nutritional function of supplying nutrients. The dietary fiber compositions of the present invention in the form of functional foods further comprise at least one edible ingredient.

As functional foods, the compositions of the present invention may be provided as, but not limited to, juices, fruit drinks, carbonated beverages, milk, nutritional drinks, ice cream, breakfast cereals, biscuits, cakes, muffins, cookies, candies, bread, toppings, bread, fiber bars, soups, crackers, baby formulae, teas, salad dressings, cooking oils, cheese, butter, jellies, jams or preserves. Further, at least one edible ingredient in the functional food compositions may be any suitable carrier or edible additive. “Edible ingredient” or “edible additive” according to the present invention includes, but is not limited to, acidulants, antioxidants, sequestrants, colors, color retention agent, sweeteners, emulsifiers, fats, oils, flavors, flavor enhancers, flour, flour treatment agents, gums, preservatives, stabilizers, spices, thickeners, bulking agents, vitamins, anticaking agents, antifoaming agents or humectants. Representative examples of each of these ingredients include, but are not limited to, acidulants such as, for example, citric acid, phosphoric acid, lactic acid, malic acid, tartaric acid, adipic acid, succinic acid, acetic acid, ascorbic acid; antioxidant agent such as, for example, glutathione, vitamin C, Melatonin, vitamin E, catalase, superoxide dismutase, peroxidases; sequestrant such as, for example, calcium disodium ethylene diamine tetra-acetate, glucono delta-lactone, sodium gluconate, potassium gluconate, sodium tripolyphoshate; food colours such as, for example, caramel, annatto, a green dye made from chlorella algae, cochineal, beet juice, turmeric, saffron, paprika; colour retention agents such as, for example, ascorbic acid, nicotinic acid; sweetners such as, for example, maple syrup, sugar beet syrup, corn syrup, cane syrup, molasses acesulfame potassium, alitame, aspartame, cyclamate, saccharin, stevioside, sucralose, inulin; emulsifiers such as, for example, egg yolk (lecithin); flavours such as, for example, diacetyl isoamyl acetate, cinnamic aldehyde, ethyl propionate, limonene, ethyl-(E,Z)-2,4-decadienoate, allyl hexanoate, ethyl maltol, methyl salicylate, benzaldehyde; flavour enhancers such as, for example, monosodium glutamate, monopotassium glutamate, calcium diglutamate, monoammonium glutamate, magnesium diglutamate, glutamic acid, guanylic acid, disodium inosinate, maltol, ethyl maltol, glycine and its sodium salt; gums such as, for example, gum arabic, gellan gum, guar gum, locust bean gum, xanthan gum; preservatives such as, for example, calcium propionate, sodium nitrate, sodium nitrite, sulfur dioxide, sodium bisulfite, potassium hydrogen sulfite, disodium EDTA; stabilizers such as, for example, gelatin, carrageenan; spices such as, for example, parsley, rosemary, coriander, berberis, mustard, black pepper, garlic, onion, ginger; thickeners such as, for example, starches, vegetable gums, and pectin, proteins; bulking agents such as, for example, starch; vitamins; anticaking agent such as, for example, sodium bicarbonate, sodium ferrocyanide, potassium ferrocyanide, sodium silicate, silicon dioxide, calcium silicate, magnesium trisilicate, talcum powder, sodium aluminosilicate, sotassium aluminium silicate, calcium aluminosilicate, bentonite, aluminium silicate, stearic acid, polydimethylsiloxane; antifoaming agent such as, for example, silicone oil; humectants like glycerine, propylene glycol, glyceryl triacetate, sorbitol, xylitol, maltitol, polydextrose; flours such as, for example, wheat flour, corn flour; flour treatment agents such as, for example, azodicarbonamide, carbamide, potassium bromate, ascorbic acid, phosphates, malted barley.

Advantageously, the functional foods of the present invention comprising effective amounts of fenugreek dietary fibers may be included in a low calorie, low fat, low cholesterol or otherwise restricted dietary regime. The compositions of the present invention provide functional foods with low glycemic index. These compositions in combination with conventional therapeutics may be used for promoting satiety and weight loss, lowering cholesterol, controlling blood glucose, maintaining colon health and modulating inflammation. Such combination therapies advantageously utilize lower dosage of the conventional therapeutics and also lower the toxicities of those agents when used alone.

In a further embodiment, compositions of the present invention additionally comprise dietary fibers from sources such as, but not limited to, oat bran, oat rim, barley, psyllium husk, soy fiber, sesame flour, flaxseed, indigestible dextrin, beet fiber, rice bran, guar gum, pectin, locust bean gum, xanthan gum, lucerne, tara gum, palm fiber, apple, banana, blackberries, dates, orange, pear, pineapple, raisins, strawberries, prunes, carrots, potato or sweet potato.

A still further embodiment of the present invention provides a method of treating or preventing disease conditions and maintaining overall health of an individual including promoting satiety and weight loss, lowering cholesterol, controlling blood glucose, maintaining colon health and modulating inflammation, by administering to the subject in need thereof compositions of the present invention comprising an effective amount of fenugreek dietary fibers.

While the present invention has been described in terms of its specific embodiments, certain modifications and equivalents will be apparent to those skilled in the art and are intended to be included within the scope of the present invention.

The present invention is further described by the following examples, which are meant solely for illustrative purposes and should not be construed as limiting the scope of the invention in any way.

### EXAMPLES

### Example 1

Comparative evaluation of water holding capacity of fenugreek dietary fiber of the present invention, soluble fiber enriched fenugreek extract and microcrystalline cellulose.

The water holding capacity of fiber is a measure of the ability of a fiber source to immobilize water within its matrix. The centrifugation and filtration method was used to compare the water holding capacity of fenugreek fiber employed in the compositions of the present invention, soluble fiber enriched fiber fenugreek extract with a ratio of insoluble to soluble fibers of 0.04 and microcrystalline cellulose, a representative of insoluble fibers. For the centrifugation method tared centrifuge tubes each containing the specified amounts of dry fiber soaked for 24 h in distilled water were centrifuged at 10000 rpm for 20 min and the supernatant fraction was decanted. The fresh weight of fiber was determined and the water holding capacity was calculated as g water/g fiber. The results obtained as depicted beneath clearly indicate that fenugreek dietary fiber has a better water holding capacity than the soluble fiber enriched extract that is commercially available from Ceejay Healthcare Private Limited and microcrystalline cellulose.

| | Water Holding Capacity gm |
|---|---|
| Sample | water/gm fiber |
| Soluble fiber enriched | 22.97 |
| fenugreek extract | |
| Microcrystalline cellulose | <2 |
| Fenugreek dietary fiber of | 26.375 |
| the present invention | |

### Example 2

Comparative evaluation of swelling index of fenugreek dietary fibers, soluble fiber enriched extract and microcrystalline cellulose.

The swelling index is the volume in millilitres occupied by 1 gram of test material after it has swollen in an aqueous liquid for 4 h. The swelling index of fenugreek fibers employed in the present invention was compared against that of soluble fiber enriched fiber fenugreek extract with a ratio of insoluble to soluble fibers of 0.04 and microcrystalline cellulose, a representative of insoluble fibers using the method laid down in European Pharmacopoeia 6.0. The method involved placing 1.0 g of the test material in a 25 ml ground-glass stoppered cylinder, which are shaken vigorously every 10 minutes for 1 hour and then allowed to stand for 3 hours. The volume occupied by the test material is measured, including any adhering mucilage. The results as depicted in the table beneath clearly indicate that the swelling index of fenugreek fibers is better than the soluble fiber enriched extract that is commercially available that is commercially available from Ceejay Healthcare Private Limited and microcrystalline cellulose.

| | Experimental Swelling Index |
|---|---|
| | (swelling volume in ml per |
| Sample | 1.0 gm of sample) |
| Soluble fiber enriched | 6.5 |
| fenugreek extract | |
| Microcrystalline | <5 |
| cellulose | |
| Fenugreek dietary fiber | 22.2 |
| of the present invention | |

Thus, fenugreek dietary fibers employed have both high water holding and swelling capacity unique to the present invention compared to both soluble dietary fiber rich extract as well as insoluble dietary fibers.

### Example 3

### Comparative Evaluation of Bile Adsorption Capacity of Fenugreek Dietary Fibers, Soluble Fiber Enriched Fenugreek Extract and Microcrystalline Cellulose

The adsorption ability of bile salts of fenugreek fibers, soluble fiber enriched fenugreek extract and microcrystalline cellulose has been investigated in vitro.

These investigations showed that the fenugreek dietary fiber employed in the compositions of the present invention had a high ability to adsorb bile salt. The fenugreek dietary fiber employed in the invention adsorbed sodium taurocholate to a significantly greater extent than was the case with soluble fiber enriched fenugreek extract available from Ceejay Healthcare Private Limited and microcrystalline cellulose.

The results show that the fenugreek dietary fibers employed in the compositions of the present invention adsorb sodium taurocholate to a greater extent that the commercially available fenugreek soluble fiber enriched extract and microcrystalline cellulose. The mechanism behind the positive effects of the dietary fiber composition embodying the invention on lowering cholesterol may be assumed to be due the better bile salt adsorption ability of the fenugreek dietary fibers employed in the present invention, which can be said to result in increased decomposition of cholesterol in the bile acid synthesis.

### Example 4

### Chewable Tablets of Fenugreek Dietary Fiber

a. Granulation of Fenugreek Dietary Fiber

| Ingredients | gm/unit |
|---|---|
| Fenugreek dietary fiber | 1.2 |
| PEG 4000, USP | 0.6 |
| Eudragit EPO, USP | 0.2 |
| Mannitol, USP | 0.6 |
| Total weight | 2.6 |

### Procedure:

The preweighed quantity of fenugreek dietary fiber was thoroughly mixed with dry blend of PEG 4000 and mannitol. This was followed by wet granulation using Eudragit EPO solution in ethanol. The granules were dried and sieved.

b. Compression of Fenugreek Dietary Fiber Granules

| Ingredients | gm/unit |
|---|---|
| Fenugreek dietary fiber granules | 2.6 |
| Citric acid, USP | 0.01 |
| Colloidal silicon dioxide, USP | 0.05 |
| Acesulfame K, Ph. Eur. | 0.2 |
| Sucralose, USP | 0.2 |
| Aspartame, USP | 0.1 |
| Orange flavor | 0.03 |
| Iron oxide red, Ph. Eur. | 0.01 |
| Iron oxide yellow, Ph. Eur. | 0.01 |
| Magnesium stearate, USP | 0.05 |
| Total weight | 3.26 |

### Procedure:

All the ingredients other than magnesium strearate were sieved and mixed with fenugreek dietary fiber granules in a polybag to prepare a uniform blend. To this blend, magnesium stearate was added and the mixture was uniformly blended again. This blend was compressed to form chewable tablets.

The chewable tablet of fenugreek dietary fiber was good to taste and palatable.

### Example 5

### Chewable Tablets of Fenugreek Dietary Fiber

| Ingredients | gm/unit |
|---|---|
| Fenugreek dietary fiber | 1.0 |
| Spray dried calcium silicate and | 1.0 |
| mannitol | |
| Mannitol, USP | 0.1 |
| Aspartame, USP | 0.05 |
| Sucralose, USP | 0.1 |
| Strawberry flavor | 0.02 |
| Magnesium stearate, USP | 0.02 |
| Total weight | 2.3 |

### Procedure:

All the ingredients except magnesium stearate were sieved and mixed with fenugreek dietary fiber in a polybag. This blend was lubricated using magnesium silicate. The blend was directly compressed to get chewable tablet of fenugreek dietary fiber. The chewable tablet imparted good mouth feel.

### Example 6

### Chewable Tablet of Fenugreek Dietary Fiber

a. Granulation of Fenugreek Dietary Fiber

| Ingredients | gm/unit |
|---|---|
| Fenugreek dietary fiber | 1.0 |
| β-cyclodextrin, USP | 0.3 |
| Glyceryl behenate, USP | 1.5 |
| Total weight | 2.8 |

### Procedure

The Fenugreek dietary fiber was blended well with β-cyclodextrin in a dry state and this was mixed with molten glyceryl behenate till a wet paste was formed. The mixture was cooled and sieved.

b. Composition of Chewable Tablet

| Ingredients | gm/unit |
|---|---|
| Fenugreek dietary fiber granules | 2.8 |
| Acesulfame K, Ph. Eur. | 0.2 |
| Sucralose, USP | 0.1 |
| Aspartame, USP | 0.2 |
| Strawberry flavor | 0.02 |
| Iron oxide red, Ph. Eur. | 0.01 |
| Iron oxide yellow, Ph. Eur. | 0.01 |
| Magnesium stearate, USP | 0.08 |
| Total weight | 3.42 |

### Procedure:

All the ingredients other than magnesium stearate were sieved and blended well in a polybag. The granules were lubricated and compressed into tablets. The tablets had good mouth feel and palatability.

### Example 7

### Stick Formulation of Fenugreek Dietary Fiber

a. Granulation of Fenugreek Dietary Fiber

| Ingredients | gm/unit |
|---|---|
| Fenugreek dietary fiber | 1.0 |
| Glyceryl behenate, USP | 1.5 |
| Mannitol, USP | 0.3 |
| Copovidone, Ph. Eur. | 0.01 |
| Total weight | 2.81 |

### Procedure:

Fenugreek dietary fiber was dispersed well in molten glyceryl behenate and cooled. Mannitol was added to the mixture and blended. Copovidone was dissolved in solvent and solution was added to the blend and granulated. The granules were dried and sieved.

| Ingredients | gm/unit |
|---|---|
| Sized granules | 2.8 |
| Colloidal silicon dioxide, USP | 0.09 |
| Acesulfame K, Ph. Eur. | 0.2 |
| Sucralose, USP | 0.1 |
| Aspartame, USP | 0.2 |
| Strawberry flavor | 0.02 |
| Iron oxide red, Ph. Eur. | 0.01 |
| Iron oxide yellow, Ph. Eur | 0.01 |
| Total weight | 3.43 |

### Procedure

Fenugreek dietary fibers were mixed with other sieved excipients in a polybag to prepare a uniform blend. The granules were filled in speciality elongated polybag containers to result in stick shape.

### Example 8

### Preparation of Fenugreek Dietary Fiber Cookies

| Ingredients | % by wt of Dough |
|---|---|
| Refined Wheat flour | 50 |
| Fenugreek dietary fiber | 15 |
| Cooking oil | 12 |
| Sucralose, USP | 10 |
| Sodium bicarbonate, USP | 0.20 |
| Water | 12 |
| Nutmeg | 0.30 |
| Vanilla flavor, USP | 0.20 |
| Butter flavor | 0.30 |
| Total | 100 |

### Procedure:

a. Sucralose was added to water and mixed well in blender for two minutes.
b. Further fenugreek dietary fiber was added to mixture and blended for 2 minutes. Edible oil was added to above mass.
c. Sodium bicarbonate was passed through sieve and other ingredients like vanilla flavor, butter flavor and nutmeg were added to sieved sodium bicarbonate. This mixture was added to part b and mixed well.
d. Refined wheat flour was sieved and was added to part c and mixed well to get dough.
e. The dough so obtained was put in the tray and filled in the moulds of dimensions 6×4×0.5 mm and baked in oven at 160 to 180° C. for 7 to 8 minutes.
f. The cookies were cooled for 10 minutes.

These cookies are convenient to carry and palatable for use.

### Example 9

### Preparation of Fenugreek Dietary Fiber Biscuits

| Ingredients | % by wt of Dough |
|---|---|
| All purpose flour | 48 |
| Fenugreek dietary fiber | 18 |
| Erythritol | 10 |
| Water | 23 |
| Sodium bicarbonate, USP | 0.7 |
| Vanilla Flavor, USP | 0.3 |
| Total | 100 |

### Procedure:

Dough was prepared by mixing the specified weighed amount of ingredients and then the mixture was blended for four minutes using a mixer at slow speed. The dough was pressed into a sheet form having dimensions of about 300 mm×500 mm×2 mm by a sheet roller. This was cut into rectangular pieces having dimensions of 10 mm×120 mm and baked in a gas oven at temperature of 180° C. to 210° C. for 8 to 15 minutes to yield fenugreek dietary fiber biscuits.

### Example 10

### Fenugreek Dietary Fiber Biscuits

| Ingredients | % by wt of Dough |
|---|---|
| All purpose flour | 50 |
| Fenugreek dietary fiber | 15 |
| Isomalt | 12 |
| Vegetable fat | 7 |
| Skimmed milk powder | 4 |
| Water | 11 |
| Ammonium bicarbonate | 0.3 |
| Sodium bicarbonate, USP | 0.4 |
| Salt | 0.3 |
| Total | 100 |

### Procedure:

a. Vegetable fat and isomalt were mixed well
b. Salt was dissolved in water
c. Part a was mixed with part b
d. Flour was mixed with sodium bicarbonate and ammonium bicarbonate and skimmed milk powder.
e. Part d was mixed with rest of the mixture
f. The dough was molded in biscuit form
g. The biscuits were baked at 225° C. for 6 minutes.

### Example 11

### Fenugreek Dietary Fiber Snack Bar

| Ingredients | % by wt of Dough |
|---|---|
| Refined wheat flour | 30 |
| Fenugreek dietary fiber | 15 |
| Corn flour | 5 |
| Coconut | 10 |
| Cashew nuts | 9 |
| Honey | 10 |
| Sugar | 8 |
| Vegetable oil | 0.3 |
| Salt | 0.3 |
| Water | 12 |
| Vanilla flavor | 0.4 |
| Total | 100 |

### Procedure:

a. Refined what flour, corn flour and fenugreek dietary fiber was passed through sieve and blended well with coconut and crushed cashew nuts.
b. Honey was mixed well with water followed by addition of sugar which was dissolved by blending. To this solution vegetable oil was added and blended for another 20 minutes to make an emulsion. Salt was added to the solution and vanilla flavor was mixed well with the above system.
c. Part b was added to part a and mixed well to form a dough.
d. The dough was molded by putting in greased container and baked at 325° F. for 25 minutes.
e. The preparation was allowed to cool and cut to give snack bars.

1) A dietary fiber composition comprising an effective amount of fenugreek dietary fiber wherein said fenugreek dietary fiber comprises soluble dietary fiber and insoluble dietary fiber.

2) The composition of claim 1 wherein said fenugreek dietary fiber is present in an amount of about 5% to about 95% by weight of said composition.

3) The composition of claim 1 wherein said soluble dietary fiber is present in an amount of about 5% to about 95% by weight of said fenugreek fiber.

4) The composition of claim 1 wherein said insoluble dietary fiber is present in an amount of about 5% to about 95% by weight of said fenugreek fiber.

5) The composition of claim 1 wherein said fenugreek fiber has a ratio of insoluble to soluble dietary fibers of between about 0.2 to about 5.

6) The composition of claim 5 wherein said fenugreek fiber has a ratio of insoluble to soluble dietary fibers of between about 0.8 to about 3.

7) The composition of claim 6 wherein said fenugreek fiber has a ratio of insoluble to soluble dietary fibers of between about 1 to about 3.

8) The composition of claim 1 wherein said composition is a dietary supplement, functional food or pharmaceutical preparation.

9) The composition of claim 8 wherein said dietary supplement or said pharmaceutical preparation further comprises at least one pharmaceutically acceptable excipient, said excipient being a filler, binder, lubricant, stabilizer, solubilizer or disintegrant.

10) The composition of claim 8 wherein said functional food further comprises at least one edible additive, said additive being an acidulant, antioxidant, sequestrant, color, color retention agent, sweetener, emulsifier, fat, oil, flavor, flavor enhancer, flour, flour treatment agent, gum, preservative, stabilizer, spice, thickener, bulking agent, vitamin, anticaking agent, antifoaming agent or humectant.

11) A method of promoting overall health and well-being of a subject comprising administering to said subject in need thereof the dietary fiber composition of claim 1 whereby at least one health benefit achieved is promotion of satiety and weight loss, lowering cholesterol, controlling blood glucose, maintaining colon health or modulating inflammation.
